(19) United States
(12) Patent Application Publication
(10) Pub. No.: US 2015/0252359 A1
(43) Pub. Date: Sep. 10, 2015

(54) Method for tracking test sample by second-generation DNA sequencing technology and detection kit

(71) Applicant: Berry Genomics Co., Ltd, Beijing (CN)

(72) Inventors: Di Chen, Beijing (CN); Zhuo Song, Beijing (CN); Jianguang Zhang, Beijing (CN); Daixing Zhou, Beijing (CN); Yang Gao, Beijing (CN)

(21) Appl. No.: 14/346,413

(22) PCT Filed: Nov. 12, 2012
(86) PCT No.: PCT/CN2012/084988

§ 371 Date: Mar. 21, 2014

(51) Int. Cl. C12N 15/10; C12Q 1/68

(52) U.S. Cl. CPC C12N 15/1065; C12Q 1/6806

(57) ABSTRACT

The disclosure claims a method for tracking a sample in a second-generation Deoxyribonucleic acid (DNA) sequencing technology and a detection kit, wherein the method includes the following steps of: 1) incorporating DNA molecular tag with a known sequence into a sample, and obtaining a sequencing sample; 2) sequencing the sequencing sample; 3) screening the molecular tag sequence from the sequencing result of step 2), and comparing with the known sequence of the molecular tag. As the sequencing process of the tag is synchronously implemented during the sequencing process of the DNA molecular, this method can be conveniently operated, and the confusion of the samples caused by manual operation can be found instantly; thereby, this method not only has important significance for the technical research, but also greatly improves the strictness of the clinical detection if applied to the clinical detection.

### TECHNICAL FIELD OF THE INVENTION

The disclosure relates to the clinical detection field, and in particular to a method for tracking a test sample by a second-generation Deoxyribonucleic acid (DNA) sequencing technology and a detection kit.

### BACKGROUND OF THE INVENTION

With the development of the sequencing technology, the traditional Sanger sequencing cannot fully satisfy the needs of the research; the second-generation sequencing technology which has lower cost, higher throughput, faster speed, and can complete the whole genome sequencing is emerged at the right moment. The core idea of the second-generation sequencing technology is to synchronously implement synthesis and sequencing with high throughput, namely, to determine the DNA sequence by catching the marker of the newly-synthesized end; the existing technical platform mainly includes Roche/454 FLX, Illumina/Genome Analyzer/Hiseq/Miseq, Applied Biosystems SOLID, and life Technologies/Ion Torrent and the like. Taking the Illumina product as an example, the sequencing throughput of 6 human genomes with 30× coverage can be reached by operating HiSeq 2000 for once at present, it generate about 600 G data during one-time operation, and the operation time of sequencing is reduced to 30 min. In addition, as the second-generation sequencing technology becomes more mature, it is rapidly developed to be applied in clinical research. Studies show that the fetus genetic health condition can be judged by sequencing the plasma DNA of the pregnant woman; and the early cancer screening can be implemented by sequencing the plasma DNA of the subject, thus the second-generation sequencing technology has a strong application prospect.

However, with the popularity of plasma DNA detection, processes of the sample detection are increased, quite a few manual operations are involved, and the probability of confusion of the samples is gradually increased when intensively detecting a large amount of samples, it becomes more and more important to track the samples and to find the confusion of samples immediately. There is no effective method to resolve the problem of confusion of the samples during the plasma/blood detection process at present.

### SUMMARY OF THE INVENTION

The disclosure aims at providing a method for tracking a test sample by a second-generation DNA sequencing technology and a detection kit, in order to solve the problem that the test samples are easy to be confused during the manual operation. In the prior art, this confusion cannot be immediately found during the sequencing process

In order to realize the above purpose, the disclosure provides a method for tracking a test sample which will be detected by a second-generation DNA sequencing technology according to one aspect. The method includes the following steps of: 1) incorporating a DNA molecular tag with a known sequence into the test sample, and obtaining a sequencing sample; 2) sequencing the sequencing sample; 3) screening the molecular tag sequence from the sequencing result of step 2), and comparing with the known sequence of the molecular tag.

Further, the DNA molecular tag is a DNA sequence with less than 20% the test DNA similarity within the sequencing range.

Further, the test sample is a blood and/or plasma sample of human; the DNA molecular tag is a DNA sequence of exogenous species or an artificial DNA with less than 20% the human genomic DNA similarity within the sequencing range; the length of the DNA molecular tag is 120-200 bp.

Further, before step 1), the method further includes: phosphorylating the 5′ terminus of the DNA molecular tag; and/or pre-phosphorylating the 5′ terminus of an amplified primer of the DNA molecular tag.

Further, the proportion of the DNA molecular tag incorporated into the blood is 1 pg-1000 pg:1 ml blood; and the proportion of the DNA molecular tag incorporated into the plasma is 0.1 pg-1000 pg:1 ml plasma.

The disclosure provides a detection kit of a test sample which will be detected by the second-generation DNA sequencing technology according to another aspect. The kit includes: a DNA molecular tag with a known sequence, a sequencing primer of a test DNA, and a sequencing primer of the DNA molecular tag.

Further, the DNA molecular tag is a DNA sequence with less than 20% the test DNA similarity within the sequencing range.

Further, the test sample is the blood and/or plasma sample of human; the DNA molecular tag is a DNA sequence of the exogenous species or an artificial DNA with less than 20% the human genomic DNA similarity within the sequencing range.

Further, the length of the DNA molecular tag is 120-200 bp. As the length of the plasma DNA fragments are approximately 166 bp, the length of the DNA molecular tag needs to be matched therewith.

Further, the kit includes: a phosphorylation reagent for phosphorylating the 5′ terminus of the DNA molecular tag; and/or a pre-phosphorylation reagent for pre-phosphorylating the 5′ terminus of the amplified primer of the DNA molecular tag.

By adopting the technical solution of the disclosure, the DNA molecular tag with the known sequence is incorporated into the test samples which will be detected by the second-generation DNA sequencing technology; and then the molecular tag sequence in the sequencing result is compared with the known sequence of the molecular tag, to judge whether the test samples are confused or not. As the sequencing process of this tag is synchronously implemented during the sequencing process of the test DNA molecules, this method can be conveniently operated, and can immediately find the confusion of the test samples caused by manual operation; thus, this method not only has important significance for the scientific research, but also greatly improves the strictness of the clinical detection if applied to the clinical detection.

### BRIEF DESCRIPTION OF THE DRAWINGS

The specifications and drawings are used for further understanding the disclosure, and forming one part of the disclosure; the exemplary embodiments of the disclosure and the descriptions thereof are used for explaining the disclosure, without improperly limiting the disclosure. In the drawings:

FIG. 1 shows a flowchart of tracking the plasma/blood test samples according to an embodiment of the disclosure;

FIG. 2 shows a flowchart of preparing the DNA molecular tags according to an embodiment of the disclosure;

FIG. 3A shows a gel electrophoretogram of the DNA molecular tags according to an embodiment of the disclosure; and

FIG. 3B shows a gel electrophoretogram of a Polymerase Chain Reaction (PCR) amplification product according to an embodiment of the disclosure.

### DETAILED DESCRIPTION OF THE EMBODIMENTS

It should note that, the embodiments of the disclosure and the characteristics in the embodiment can be mutually combined without conflict. The disclosure is described as below with reference to the drawings and embodiments in details.

The disclosure provides a method for tracking a test sample by a second-generation DNA sequencing technology according to a typical embodiment. The method includes the following steps of: 1) incorporating a DNA molecular tag with a known sequence into the test sample, and obtaining a sequencing sample; 2) sequencing the sequencing sample; 3) screening the molecular tag sequence from the sequencing result of step 2), and comparing with the known sequence of the molecular tag. If the information of the molecular tag sequence in the sequencing result is matched with the information of the corresponding molecular tag incorporated into the test sample, indicating that there is no wrongly marked samples and cross-contamination. As the sequencing process of this tag is synchronously implemented during the sequencing process of the test DNA molecules, this method can be conveniently operated, and can immediately find the confusion of the test samples caused by manual operation; thus, this method not only has important significance for the scientific research, but also greatly improves the strictness of the clinical detection if applied to the clinical detection.

The lower similarity between the DNA molecular tag and the test DNA sequence within the sequencing range is, the higher detection analysis accuracy and speed are. Preferably, the DNA molecular tag is a DNA sequence with less than 20% the test DNA similarity within the sequencing range, thus, the DNA molecular tag sequence can be rapidly distinguished from the sequence of the test DNA molecules in the sequencing result, so as to improve the detection analysis speed and to make the analysis of batch samples to be convenient.

According to one typical embodiment of the disclosure, the test sample is the blood and/or plasma sample of human, the DNA molecular tag is a DNA sequence of exogenous species or an artificial DNA with less than 20% the human genomic DNA similarity within the sequencing range. If the technical solution of the disclosure is applied to the clinical detection, the obtained effect can be more obvious. As in consideration of the strictness of the clinical detection, the false negative- and false-positive caused by confusion of samples need to be found and removed fundamentally; by adopting the technical solution of the disclosure, a certain proportion of exogenous DNA (DNA molecular tag) is added into the plasma and/or blood, and via the second-generation sequencing technology, the exogenous DNA sequence is compared with the record of incorporated DNA molecular tag without influencing the detection; the confusion of samples can be determined if the sample record is inconsistent with the actual incorporation. As the length of the plasma DNA fragment is approximately 166 bp, in order to make sequencing be convenient after constructing the library, the length of the exogenous DNA which is incorporated when detecting the plasma and/or blood sample is 120-200 bp, preferably, 166±10 bp, wherein, the exogenous DNA can be selected from other species which have poor homology with the human genome, or can be artificial.

In order to make high-efficient sequencing be convenient, preferably, before step 1), the method further includes: phosphorylating the 5′ terminus of the DNA molecular tag; and/or pre-phosphorylating the 5′ terminus of an amplified primer of the DNA molecular tag, which is beneficial for improving the efficiency of TA cloning.

Preferably, the proportion of the DNA molecular tag incorporated into the blood is 1 pg-1000 pg:1 ml blood; and the proportion of the DNA molecular tag incorporated into the plasma is 0.1 pg-1000 pg:1 ml plasma; such proportion of addition amount can make the molecular tag detection effective and accurate without influencing the fast and effective sequence detection of the test DNA molecule. It adopts the second-generation sequencing technology in step 2), because the second-generation sequencing technology not only has high sequencing throughput, but also has accurate result, which is quite suitable for being applied in the technical solution of the disclosure.

The disclosure provides a detection kit of a test sample which will be detected by the second-generation DNA sequencing technology according to a typical embodiment. The detection kit includes: a DNA molecular tag with a known sequence, a sequencing primer of a test DNA, and a sequencing primer of the DNA molecular tag. By using this kit, the strictness of detection can be greatly improved by applying the technical solution of the disclosure to detect the test samples in the second-generation DNA sequencing technology.

Preferably, the DNA molecular tag is a DNA sequence with less than 20% the test DNA similarity within the sequencing range, thus the sequence of the DNA molecular tag can be rapidly distinguished from the sequence of the test DNA molecules in the sequencing result, so as to improve the detection analysis speed and to make the analysis of batch samples be convenient.

Preferably, the test sample in the second-generation DNA sequencing technology is the blood and/or plasma sample of human; the DNA molecular tag is the DNA of the exogenous species or an artificial DNA with less than 20% the human genomic DNA similarity within the sequencing range. As in consideration of the strictness of the clinical detection, the false-negative and false-positive caused by confusion of samples need to be found and removed fundamentally; by adopting the technical solution of the disclosure, a certain proportion of exogenous DNA (DNA molecular tag) is added into the plasma and/or blood, and via the second-generation sequencing technology, the exogenous DNA sequence is compared with the record of incorporated DNA molecular tag without influencing the detection; the confusion of samples can be determined if the sample record is inconsistent with the actual incorporation. As the length of the plasma DNA fragment is approximately 166 bp, the length of the exogenous DNA which is incorporated when detecting the plasma and/or blood sample is 120-200 bp, preferably, 166±10 bp, wherein, the exogenous DNA can be selected from other species which have poor homology with the human genome, or can be artificial.

Preferably, the kit further includes: a phosphorylation reagent for phosphorylating the 5′ terminus of the DNA molecular tag; and/or a pre-phosphorylation reagent for pre-phosphorylating the 5′ terminus of the amplified primer of the DNA molecular tag.

The beneficial effects of the disclosure are further described with reference to the embodiments.

### Embodiment

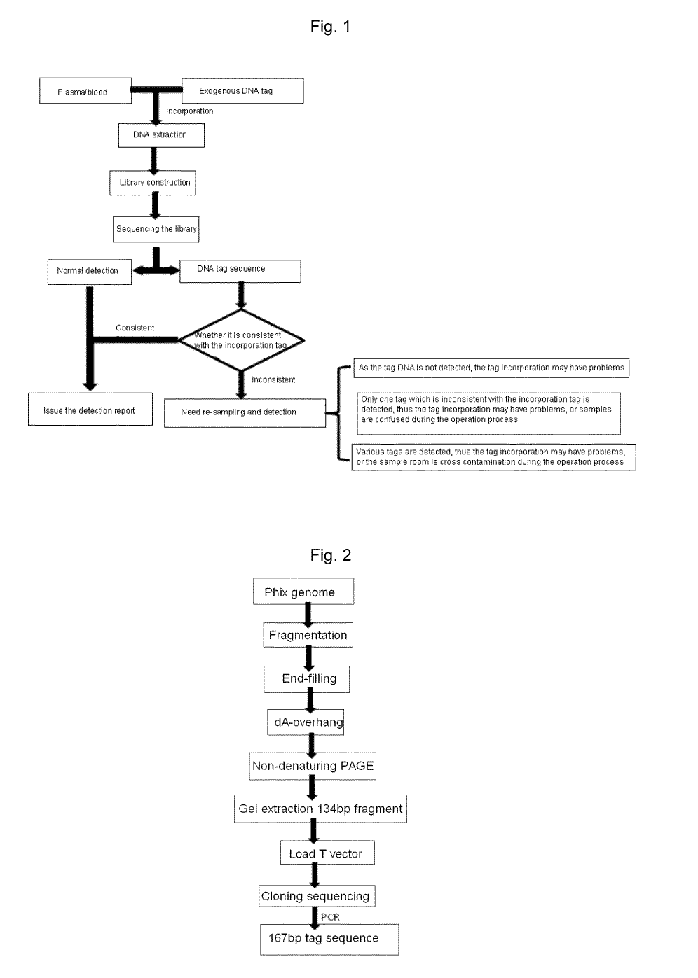

The flow of tracking the plasma/blood test sample of the embodiment is as shown in FIG. 1.

This embodiment includes the steps of: fragmenting the phix fragments of exogenous genome which have poor homology with human, selecting the fragments with certain length, obtaining a single phix fragment sequence via TA cloning, and determining the sequence formation via Sanger sequencing; the amplified phix fragment from plasmid DNA by PCR with corresponding length of approximate 167 bp serves as a molecular tag. The flow of preparing the DNA molecular tag is as shown in FIG. 2. Obviously, the exogenous genomic DNA adopted by this embodiment is the phix genome, but is not limited by phix genome, any genome which has poor homology with human can be taken as the molecular tag, for example, an artificially designed and synthesized sequence can be taken as the molecular tag.

The reagent and operation steps adopted by this embodiment are as follows:

1. The TA cloning vector is TAKARA pMD19-T Vector, the amplified primers for plasmid DNA are Barcode-F and Barcode-R, and the 5′ terminus of the primer needs to be phosphorylated:

Barcode-F:
5′ pCCGGGGATCCTCTAGAGAT 3′
Barcode-R:
5′ pATGCCTGCAGGTCGACGAT 3′

The structure of the DNA tag sequence:

5′ ATGCCTGCAGGTCGACGATTNNN...
NNNAATCTCTAGAGGATCCCCGG 3′
3′ TACGGACGTCCAGCTGCTAANNN...
NNNTTAGAGATCTCCTAGGGGCC 5

The sequence of the DNA tag 1:

ATGCCTGCAGGTCGACGATTCAGTAAGAACGTCAGTGTTTCCTGCGCGTA
CACGCAAGGTAAACGCGAACAATTCAGCGGCTTTAACCGGACGCTCGACG
CCATTAATAATGTTTTCCGTAAATTCAGCGCCTTCCAATCTCTAGAGGAT
CCCCGG

The sequence of the DNA tag 2:

ATGCCTGCAGGTCGACGATTGTCCTGCGTGTAGCGAACTGCGATGGGCAT
ACTGTAACCATAAGGCCACGTATTTTGCAAGCTATTTAACTGGCGGCGAT
TGCGTACCCGACGACCAAAATTAGGGTCAACGCTACCTAATCTCTAGAGG
ATCCCCGG

2. Sample

The plasma sample: taking 1 ml of normal human plasma, adding a corresponding quantity of tag DNA, extracting the free DNA in plasma.

Whole blood sample: adding 1 ml of peripheral blood of normal human, adding 5 ng of tag DNA to implement plasma separation, and extracting the free DNA in plasma.

The relationship between the sample and the incorporation quantity of tag is as shown in Table 1:

TABLE 1

| | Test | Sample | Incorporation | Incorporated |
|---|---|---|---|---|
| Number of samples | sample | quantity | quantity | tag |
| RB12X10665_2A | Plasma | 1 ml | 1 ng | tag1 |
| RB12X11601_3A | Plasma | 1 ml | 1 ng | tag 1 |
| RB12X09283_8A | Plasma | 1 ml | 1 ng | tag 2 |
| RB12X10663_9A | Plasma | 1 ml | 1 ng | tag 2 |
| RB12X17683_4A | Whole | 1 ml | 2 ng | tag1 |
| | blood | | | |
| RB12X11492_5A | Whole | 1 ml | 2 ng | tag 1 |
| | blood | | | |
| RB12X14912_6A | Whole | 1 ml | 5 ng | tag 1 |
| | blood | | | |
| RB12X17681_7A | Whole | 1 ml | 5 ng | tag 1 |
| | blood | | | |
| RB12X11590_10A | Whole | 1 ml | 2 ng | tag 2 |
| | blood | | | |
| RB12X14010_11A | Whole | 1 ml | 2 ng | tag 2 |
| | blood | | | |
| RB12X11587_12A | Whole | 1 ml | 5 ng | tag 2 |
| | blood | | | |
| RB12X17682_20A | Whole | 1 ml | 5 ng | tag 2 |
| | blood | | | |
| RB12X13648_21A | Whole | 1 ml | 10 ng | tag 2 |
| | blood | | | |
| RB12X11493_22A | Whole | 1 ml | 10 ng | tag 2 |
| | blood | | | |

3. End-filling

Preparation of the following reaction mixture

| | |
|---|---|
| DNA solution of test sample | 38.5 μl |
| T4 DNA phosphorylation buffer (10X) | 5 μl |
| 10 mM dNTP mixture | 2 μl |
| T4 DNA polymerase | 2 μl |
| T4 DNAphosphorylase | 2 μl |
| Klenow enzyme | 0.5 μl |
| Sterile H₂O | 0 μl |
| Total volume | 50 μl |

a. Incubating for 30 min at 20 degrees centigrade;

b. purifying the DNA samples by a purification column, eluting the samples with 42 μl of sterile dH₂O or elution buffer, and obtaining the blunt-ended DNA.

4. Adding polyadenylation tail at the 3′ terminus of the DNA fragment

Preparation of the following reaction mixture:

| | |
|---|---|
| Blunt-ended DNA | 32 μl |
| Klenow reaction buffer (10X) | 5 μl |
| dATP solution | 10 μl |
| klenow ex- (deletion of 3′-5′ exonuclease | 3 μl |
| activity) | |
| Sterile H₂O | 0 μl |
| Total volume | 50 μl |

a. Incubating for 30 min at 37 degrees centigrade;

b. purifying the DNA samples by the purification column, eluting the samples with 25 μl of sterile dH₂O or elution buffer, and obtaining the blunt-ended poly(dA)-tail DNA.

5. Ligating adaptor to the DNA fragment

Preparation of the following reaction mixture

| | |
|---|---|
| Blunt-ended poly(dA)-tail DNA | 33 μl |
| Fast ligation reaction buffer (5X) | 10 μl |
| 5 μM DNA adaptor | 2 μl |
| Fast T4 DNA ligase (NEB) | 5 μl |
| Total volume | 50 μl |

a. Incubating for 15 min at 20 degrees centigrade;

b. purifying and recycling the DNA samples by a Qiagen column, and eluting the samples with 25 μl of sterile dH₂O or elution buffer.

6. Enriching the adaptor-modified DNA fragment via PCR pre-amplification

Preparation of the following PCR reaction mixture

| | |
|---|---|
| DNA (obtained in Step 5) | 12.5 μL |
| Phusion DNA polymerase (Phusion DNA | 25 μl |
| polymerase mixture) | |
| PCR primer mixture | 2 μl |
| Ultrapure water | 10.5 μl |
| Total volume | 50 μl |

Amplifying via the following PCR experimental program:

a. 98 degrees centigrade for 30 s;

b. 18 cycle as follows:

98 degrees centigrade for 10 s, 65 degrees centigrade for 30 s, 72 degrees centigrade for 30 s;

c. 72 degrees centigrade for 5 min;

d. Incubating at 4 degrees centigrade.

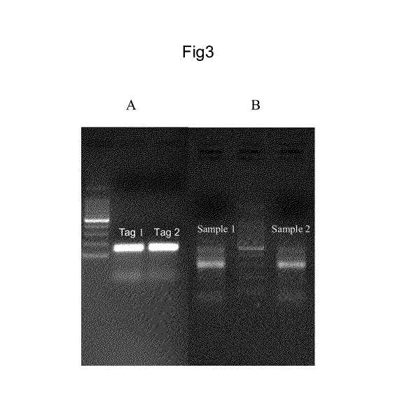

7. Loading the PCR products obtained in Step 6 on 2% of agarose gel to implement electrophoresis, the result is shown in FIG. 3b; and then extracting 300 by target bands (DNA library) by using Qiagnen kit gel extraction kit, eluting with 30 μl of elution buffer. FIG. 3a shows the gel electrophoretogram of the DNA molecular tag adopted by the embodiment of the disclosure.

8. After implementing quality control for the constructed library, executing 36 bp single-end sequencing by the Illumina Hiseq 2000 system.

The sequencing analysis results are shown in Table 2.

TABLE 2

| | | | | | DNA sequence | Tag 1 | Tag 2 | |
|---|---|---|---|---|---|---|---|---|
| | Status of | Sample | Incorporated | Incorporation | of the samples | (copy | (copy | |
| Numbers of sample | sample | quantity | quantity | tag | (copy number) | number) | number) | Tag (%) |
| RB12X10665_2A | Plasma | 1 ml | 1 ng | Tag 1 | 6216158 | 27558 | 0 | 0.443% |
| RB12X11601_3A | Plasma | 1 ml | 1 ng | Tag 1 | 6204828 | 27546 | 0 | 0.444% |
| RB12X09283_8A | Plasma | 1 ml | 1 ng | Tag 2 | 7058416 | 0 | 30534 | 0.433% |
| RB12X10663_9A | Plasma | 1 ml | 1 ng | Tag 2 | 6680466 | 0 | 37836 | 0.566% |
| RB12X17683_4A | Whole blood | 1 ml | 2 ng | Tag 1 | 5973710 | 121687 | 0 | 2.037% |
| RB12X11492_5A | Whole blood | 1 ml | 2 ng | Tag 1 | 5937630 | 51628 | 0 | 0.870% |
| RB12X14912_6A | Whole blood | 1 ml | 5 ng | Tag 1 | 6151790 | 127048 | 0 | 2.065% |
| RB12X17681_7A | Whole blood | 1 ml | 5 ng | Tag 1 | 6473501 | 90930 | 0 | 1.405% |
| RB12X11590_10A | Whole blood | 1 ml | 2 ng | Tag 2 | 6087380 | 0 | 31186 | 0.512% |
| RB12X14010_11A | Whole blood | 1 ml | 2 ng | Tag 2 | 7786953 | 0 | 22920 | 0.294% |
| RB12X11587_12A | Whole blood | 1 ml | 5 ng | Tag 2 | 7083880 | 0 | 75421 | 1.065% |
| RB12X17682_20A | Whole blood | 1 ml | 5 ng | Tag 2 | 5184906 | 0 | 75196 | 1.450% |
| RB12X13648_21A | Whole blood | 1 ml | 10 ng | Tag 2 | 5068772 | 0 | 130200 | 2.569% |
| RB12X11493_22A | Whole blood | 1 ml | 10 ng | Tag 2 | 5750289 | 0 | 220099 | 3.828% |

Sequencing the library of the test sample, although each sample is incorporated with one kind of DNA tag, the DNA tags incorporated in other samples can be synchronously detected during the detection; if only the DNA tag incorporated in this sample is detected, and the incorporation quantity of the other DNA tags is 0, the accuracy of the sample can be better determined.

The Table 2 shows that a linear relationship exists between the tag incorporation quantity and the tags, calculated from the practical application, the proportion of the molecular tag incorporated into the whole blood is 1 pg-100 pg:1 ml whole blood; and the maximum detection efficiency can be achieved when the proportion of the molecular tag incorporated into the whole blood is 0.1 pg-10 pg:1 ml plasma. In view of the data in Table 2, the confusion of samples does not exist during the sample detection process of the embodiment.

The above is only the preferred embodiment of the disclosure, but not intended to limit the disclosure; for those skilled in the field, the disclosure can have various changes and modifications. Any modifications, equivalent replacement and improvement implemented within the spirits and principle of the disclosure shall fall within the protection scope of the disclosure.

What is claimed is:

1. A method for tracking a test sample by a second-generation Deoxyribonucleic acid (DNA) sequencing technology, wherein the method comprises the following steps of:
1) incorporating a DNA molecular tag with a known sequence into the test sample, and obtaining a sequencing sample;
2) sequencing the sequencing sample;
3) screening the molecular tag sequence from the sequencing result of step 2), and comparing with the known sequence of the molecular tag.

2. The method according to claim 1, wherein the DNA molecular tag is a DNA sequence with less than 20% the test DNA similarity within the sequencing range.

3. The method according to claim 1, wherein the test sample is a blood and/or plasma sample of human; the DNA molecular tag is a DNA sequence of exogenous species or an artificial DNA with less than 20% the human genomic DNA similarity within the sequencing range; the length of the DNA molecular tag is 120-200 bp.

4. The method according to claim 3, wherein before the step 1), the method further comprises:
phosphorylating the 5′ terminus of the DNA molecular tag; and/or
pre-phosphorylating the 5′ terminus of an amplified primer of the DNA molecular tag.

5. The method according to claim 3, wherein the proportion of the DNA molecular tag incorporated into the blood is 1 pg-1000 pg:1 ml blood; and the proportion of the DNA molecular tag incorporated into the plasma is 0.1 pg-1000 pg:1 ml plasma.

6. A detection kit of a test sample by a second-generation DNA sequencing technology, wherein the detection kit comprises: a DNA molecular tag with a known sequence, a sequencing primer of a test DNA, and a sequencing primer of the DNA molecular tag.

7. The detection kit according to claim 6, wherein the DNA molecular tag is a DNA sequence with less than 20% the test DNA similarity within the sequencing range.

8. The detection kit according to claim 6, wherein the test sample is a blood and/or plasma sample of human; the DNA molecular tag is a DNA sequence of the exogenous species or an artificial DNA with less than 20% the human genomic DNA similarity within the sequencing range.

9. The detection kit according to claim 8, wherein the length of the DNA molecular tag is 120-200 bp.

10. The detection kit according to claim 8, further including: a phosphorylation reagent for phosphorylating the 5′ terminus of the DNA molecular tag; and/or a pre-phosphorylation reagent for pre-phosphorylating the 5′ terminus of the amplified primer of the DNA molecular tag.
